(19) United States
(12) Patent Application Publication
(10) Pub. No.: US 2021/0186845 A1
(43) Pub. Date: Jun. 24, 2021

(54) VITAMIN A AND VITAMIN C COMBINATIONS WITH ENHANCED STABILITY

(71) Applicants: Barnet Products, LLC, Englewood Cliffs, NJ (US); Nikko Chemicals Company, Ltd., Tokyo (JP)

(72) Inventors: Steven Kosann, Englewood Cliffs, NJ (US); Nickolas Huss, Maywood, NJ (US); Michelle Howard, Warren, NJ (US); Yiming Lu, Weehawken, NJ (US); Hidetomo Sekine, Tokyo (JP)

(73) Assignees: Barnet Products, LLC, Englewood Cliffs, NJ (US); Nikko Chemicals Company, Ltd., Tokyo (JP)

(21) Appl. No.: 17/038,384

(22) Filed: Sep. 30, 2020

Related U.S. Application Data
(60) Provisional application No. 62/949,533, filed on Dec. 18, 2019.

(51) Int. Cl. A61K 8/67; A61Q 5/02; A61Q 5/12; A61Q 1/04; A61Q 1/00; A61Q 17/04

(52) U.S. Cl. CPC A61K 8/676; A61K 8/671; A61Q 5/02; A61Q 17/04; A61Q 1/04; A61Q 1/00; A61Q 5/12

(57) ABSTRACT

The present invention is related to a combination for obtaining improved stability of a Vitamin A ester and Vitamin C formulation, achieved via including a specific Vitamin A derivative and one of several other Vitamin C derivatives. This combination shows an enhanced stability in varied cosmetic formulations as a function of time and at different environmental conditions.

### RELATED APPLICATION

The present application claims priority to U.S. Provisional Application Ser. No. 62/949,533, filed Dec. 18, 2019, entitled “Vitamin A And Vitamin C Combination With Enhanced Stability”, which is hereby incorporated by reference in its entirety.

### FIELD OF THE INVENTION

The present invention is related to a combination for obtaining improved stability of a Vitamin A ester and Vitamin C formulation, achieved via including a specific Vitamin A derivative and one of several other Vitamin C derivatives. This combination shows an enhanced stability in varied cosmetic formulations as a function of time and at different environmental conditions.

### BACKGROUND OF INVENTION

This invention relates to cosmetic compositions which contain a specific type of Vitamin A derivative and one of several other Vitamin C derivatives. These combinations provide not only functionality to prevent skin aging, assist skin repair and control skin keratinization, but also work synergistically in cosmetic formulations. These specific combinations help to preserve both stability of Vitamin A and Vitamin C, since these active ingredients are sensitive to external stimuli, such as pH, temperature, oxygen and so on.

Retinol, also known as Vitamin A, plays an important role in assisting the normal functioning of a variety of skin processes. For example, it participates in the regulation of epidermal cell growth and enhances glycosaminoglycans synthesis. In the field of cosmetics, Vitamin A is widely used because it is a valuable active in controlling the keratinization in normal skin. However, retinol itself is very unstable in ambient environment due to its sensitivity to oxygen, heat and UV light. Long term exposure to these environmental factors does not only accelerate the decomposition of retinol, but also decreases its efficacy. In order to prevent the deterioration of retinol, chemical derivatives of retinol are synthesized and utilized with enhanced stability. Typical examples of retinol derivatives are retinyl palmitate, propionate and linoleate. Other approaches to increase the stability and efficacy of Vitamin A are using encapsulation technology (using liposome, silica nanoparticles), adding assistant antioxidant (α-tocopherol) and combining UV absorber (oxybenzone).

Ascorbic acid, also known as Vitamin C, has important physiological effects on skin. It inhibits melanogenesis, promotes the biosynthesis of collagen, and prevents formation of radicals, due to its well-known antioxidant activity. However, to formulate finished products with ascorbic acid is not ideal because ascorbic acid is unstable. When it is exposed in aerobic condition, alkaline environment or irradiated by UV/visible light, it oxidizes irreversibly to dehydroascorbic acid, which is biologically inactive.

Therefore, derivatives of Vitamin C in other forms are synthesized or produced, especially in ester form, obtained via esterification of the hydroxyl group with long chain fatty acid. Typical examples are ascorbyl palmitate and tetrahexydecyl ascorbate/ascorbyl tetraisopalmitate. These Vitamin C esters are oil and lipid soluble with higher stability. Other common water-soluble and stable Vitamin C derivatives are ascorbyl glucoside, magnesium ascorbyl phosphate and 3-O-ethyl ascorbic acid.

As stated, the original chemical forms of Vitamin A and Vitamin C (when used individually) are unstable toward environmental stimuli, and even if they are formulated in a final cosmetic product, protective antioxidants are essential to prevent the degradation of the molecules, thus preserving their biological activity. Unfortunately, the stability of retinol combined with Vitamin C is also poor, and may render color to the formulation when exposed to the ambient environment as a function of time at a certain temperature. By using the combination presented in this invention, the cosmetic formulations have much higher stability.

The combinations disclosed here provide solutions which improve the stability of a Vitamin A and Vitamin C formulation. It is achieved by using one specific Vitamin A derivative, and one of several other Vitamin C derivatives, respectively.

The combination presented in this invention is not limited or restricted to use in any cosmetic formulations with a range of suggested use levels.

### SUMMARY OF THE INVENTION

The present invention offers a solution for attaining improved stability of a Vitamin A and Vitamin C combination that contains at least two different active vitamins, which are diluted with a carrier oil or used as pure vitamins.

In one embodiment, the Vitamin A used in the combination is oil and lipid soluble. In another embodiment the Vitamin C used in the combination can be oil soluble or water soluble.

According to the invention, the carrier oil of Vitamin A is selected from the preferred embodiment of alkyl esters and hydrocarbons. Examples are soybean oil methyl or ethyl ester, linseed oil methyl or ethyl ester, coconut oil methyl or ethyl ester, castor oil methyl or ethyl ester, olive oil methyl or ethyl ester, cottonseed oil methyl or ethyl ester, glyceryl monostearate, caprylic capric triglyceride, isopropyl myristate, isopropyl palmitate, cetyl octanoate, cetyl palmitate, mineral oil and squalane.

According to one preferred embodiment, the carrier oil for Vitamin A is squalane.

According to the invention, one of the active Vitamins is selected from the group consisting of Vitamin A and Vitamin A derivatives, preferably selected from a group consisting of all-trans retinol, retinol, retinal, retinyl acetate, retinaldehyde, retinyl palmitate, retinoic acid, retinyl propionate, retinyl linoleate, dehydroretinol and hydroxypinacolone retinoate.

According to one preferred embodiment the active Vitamin A derivative is retinyl linoleate. According to the invention one of the active Vitamins is selected from the group consisting of Vitamin C and Vitamin C derivatives, preferably selected from a group consisting of ascorbic acid, 3-O-ethyl-ascorbic acid, ascorbyl glucoside, ascorbyl octanoate, ascorbyl palmitate, ascorbyl stearate, ascorbyl dipalmitate, L-dehydroascrobic acid, sodium ascorbyl phosphate, tetrahexydecyl ascorbate/ascorbyl tetraisopalmitate, or magnesium ascorbyl phosphate.

According to another preferred embodiment, the Vitamin C derivatives are 3-O-ethyl-ascorbic acid, ascorbyl glucoside, magnesium ascorbyl phosphate and tetrahexydecyl ascorbate/ascorbyl tetraisopalmitate.

The Vitamin compositions can be applied in a variety of final products in the form of a lotion, a cream, a gel, an oil, a spray, a foam, a solid stick, a shampoo, a hair conditioner, a powder, a lacquer, a make-up or a sunscreen.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1A Stability photo (Week 1) of hydrogenated-lecithin emulsions formulated with Vitamin C derivatives and retinyl linoleate. The samples are incubated at different temperature (25° C., 45° C. and 55° C.) indicated on the left of each row. The bottom label “Group Retinyl Linoleate” indicates that all samples are formulated with retinyl linoleate. Four different INCI names of Vitamin C derivatives are labeled on top of each column. For instance, the top right sample is formulated with tetrahexyldecyl ascorbate/ascorbyl tetraisopalmitate and retinyl linoleate in a hydrogenated-lecithin emulsion base, and it is incubated at 55° C. for a week. (3-O-ethyl ascorbic acid is labeled as ethyl ascorbic acid to allow space for sample display. This label is also valid for all other figures.)

FIG. 1B Stability photo (Week 1) of hydrogenated-lecithin emulsions formulated with Vitamin C derivatives and retinol. The samples are incubated at different temperature (25° C., 45° C. and 55° C.) indicated on the left of each row. The bottom label “Group Retinol” indicates that all samples are formulated with retinol. Four different INCI names of Vitamin C derivatives are labeled on top of each column. For instance, the top right sample is formulated with tetrahexyldecyl ascorbate/ascorbyl tetraisopalmitate and retinol in a hydrogenated-lecithin emulsion base, and it is incubated at 55° C. for a week.

FIG. 1C Stability photo (Week 1) of hydrogenated-lecithin emulsions formulated with Vitamin C derivatives and hydroxypinacolone retinoate. The samples are incubated at different temperature (25° C., 45° C. and 55° C.) indicated on the left of each row. The bottom label “Group Hydroxypinacolone Retinoate” indicates that all samples are formulated with hydroxypinacolone retinoate. Four different INCI names of Vitamin C derivatives are labeled on top of each column. For instance, the top right sample is formulated with tetrahexyldecyl ascorbate/ascorbyl tetraisopalmitate and hydroxypinacolone retinoate in a hydrogenated-lecithin emulsion base, and it is incubated at 55° C. for a week.

FIG. 2A Stability photo (Week 5) of hydrogenated-lecithin emulsions formulated with Vitamin C derivatives and retinyl linoleate. The samples are incubated at different temperature (25° C., 45° C. and 55° C.) indicated on the left of each row. The bottom label “Group Retinyl Linoleate” indicates that all samples are formulated with retinyl linoleate. Four different INCI names of Vitamin C derivatives are labeled on top of each column. For instance, the top right sample is formulated with tetrahexyldecyl ascorbate/ascorbyl tetraisopalmitate and retinyl linoleate in a hydrogenated-lecithin emulsion base, and it is incubated at 55° C. for 5 weeks.

FIG. 2B Stability photo (Week 5) of hydrogenated-lecithin emulsions formulated with Vitamin C derivatives and retinol linoleate. The samples are incubated at different temperature (25° C., 45° C. and 55° C.) indicated on the left of each row. The bottom label “Group Retinol” indicates that all samples are formulated with retinol. Four different INCI names of Vitamin C derivatives are labeled on top of each column. For instance, the top right sample is formulated with tetrahexyldecyl ascorbate/ascorbyl tetraisopalmitate and retinol in a hydrogenated-lecithin emulsion base, and it is incubated at 55° C. for 5 weeks.

FIG. 2C Stability photo (Week 5) of hydrogenated lecithin emulsions formulated with Vitamin C derivatives and hydroxypinacolone retinoate. The samples are incubated at different temperature (25° C., 45° C. and 55° C.) indicated on the left of each row. The bottom label “Group Hydroxypinacolone Retinoate” indicates that all samples are formulated with hydroxypinacolone retinoate. Four different INCI names of Vitamin C derivatives are labeled on top of each column. For instance, the top right sample is formulated with tetrahexyldecyl ascorbate/ascorbyl tetraisopalmitate and hydroxypinacolone retinoate in a hydrogenated-lecithin emulsion base, and it is incubated at 55° C. for 5 weeks.

FIG. 3A Stability photo (Week 8) of hydrogenated-lecithin emulsions formulated with Vitamin C derivatives and retinyl linoleate. The samples are incubated at different temperature (25° C. and 45°) indicated on the left of each row. The bottom label “Group Retinyl Linoleate” indicates that all samples are formulated with retinyl linoleate. Four different INCI names of Vitamin C derivatives are labeled on top of each column. For instance, the top right sample is formulated with tetrahexyldecyl ascorbate/ascorbyl tetraisopalmitate and retinyl linoleate in a hydrogenated-lecithin emulsion base, and it is incubated at 45° C. for 8 weeks.

FIG. 3B Stability photo (Week 8) of hydrogenated-lecithin emulsions formulated with Vitamin C derivatives and retinol. The samples are incubated at different temperature (25° C. and 45° C.) indicated on the left of each row. The bottom label “Group Retinol” indicates that all samples are formulated with retinol. Four different INCI names of Vitamin C derivatives are labeled on top of each column. For instance, the top right sample is formulated with tetrahexyldecyl ascorbate/ascorbyl tetraisopalmitate and retinol in a hydrogenated-lecithin emulsion base, and it is incubated at 45° C. for 8 weeks.

FIG. 3C Stability photo (Week 8) of hydrogenated lecithin emulsions formulated with Vitamin C derivatives and hydroxypinacolone retinoate. The samples are incubated at different temperature (25° C. and 45° C.) indicated on the left of each row. The bottom label “Group Hydroxypinacolone Retinoate” indicates that all samples are formulated with hydroxypinacolone retinoate. Four different INCI names of Vitamin C derivatives are labeled on top of each column. For instance, the top right sample is formulated with tetrahexyldecyl ascorbate/ascorbyl tetraisopalmitate and hydroxypinacolone retinoate in a hydrogenated-lecithin emulsion base, and it is incubated at 45° C. for 8 weeks.

### DETAILED DESCRIPTION OF THE INVENTION

The combination presented in this invention allows the co-formulation of at least one of the Vitamin A derivatives, and one of the several other Vitamin C derivatives, while maintaining high stability in an ambient environment or at an elevated temperature. The enhanced stability is achieved by combining the esterified Vitamins and utilizing the synergistic anti-oxidative activities of these ingredients.

In the preferred embodiment of this invention, retinyl linoleate is used as the designated ingredient as Vitamin A derivative. It is known that the basic form of Vitamin A, retinol, is extremely sensitive to oxygen and UV light due to the decomposition of conjugated double bond in the presence of a free radical. In addition, this instability makes it difficult to formulate. By using the esterified Vitamin A, retinyl linoleate, it can effectively resist the potential negative influence from the surrounding environment. A carrier oil is used to dilute retinyl linoleate, and the oil used in this invention is squalane. The amount of retinyl linoleate in squalane is in the range of 1 to 10%.

In another preferred embodiment of this invention, one of several other Vitamin C derivatives is selected as the second ingredient for this particular combination. Specifically, these Vitamin C derivatives are 3-O-ethyl ascorbic acid, magnesium ascorbyl phosphate, ascorbyl glucoside and tetrahexydecyl ascorbate/ascorbyl tetraisopalmitate. 3-O-ethyl ascorbic acid, magnesium ascorbyl phosphate and ascorbyl glucoside are water soluble and in the form of solid powder. Tetrahexyldecyl ascorbate/ascorbyl isopalmitate is oil soluble, and in the form of a liquid.

The combination revealed in the present invention with the enhanced stability can be applied to any known cosmetic skincare products, as well as to any new formulations. The benefits derived from this invention can be applied to extend the efficacy and shelf life of any cosmetic skincare product which provides functionality for skin repair, skin rejuvenation and skin protection.

In a preferred embodiment of this invention, other Vitamin A derivatives are combined with Vitamin C derivatives to create a comparison against the retinyl linoleate combination. Examples of these Vitamin A derivatives are retinol and hydroxypinacolone retinoate.

### EXAMPLES

The following non limiting examples are provided for illustration purposes in order to facilitate a better understanding of the disclosure subjects.

### List of Ingredients

The ingredients presented here may be obtained from varied suppliers and may be substituted with other ingredients sharing the same INCI name.

TABLE 1

| Active Vitamins | INCI Name |
|---|---|
| Group of Vitamin A | Retinyl Linoleate |
| Derivatives | Retinol |
| | Hydroxypinacolone Retinoate |
| Group of Vitamin C | Tetrahexydecyl |
| Derivatives | Ascorbate/Ascorbyl |
| | Tetraisopalmitate |
| | Asborbyl Glucoside |
| | 3-O-Ethyl Ascorbic Acid |
| | Magnesium Ascorbyl Phosphate |

The combination of Vitamin A and Vitamin C presented in this invention can be used in a wide range of skincare formulations. Depending on the hydrophilicity and hydrophobicity of the chosen Vitamin A or Vitamin C derivatives, the addition of these two components should comply with the specific formulation guideline. For example, a skincare formulation is often composed of several different phases. When using an oil/lipid soluble Vitamin C derivative, it is necessary to combine this ingredient first with the oil phase, and then move onto the following steps. In a preferred embodiment of this invention, the oil soluble Vitamin C derivative is tetrahexydecyl ascorbate/ascorbyl tetraisopalmitate. For water soluble Vitamin C derivatives, it is vital to combine these ingredients first in an aqueous phase, and then move onto the following steps of formulation. In another preferred embodiment of this invention, water soluble Vitamin C derivatives are ascorbyl glucoside, magnesium ascorbyl phosphate and 3-O-ethyl ascorbic acid. These three derivatives are in the form of a solid. Yet in another preferred embodiment, retinyl linoleate is the Vitamin A derivative which is used in the combination. Other Vitamin A or Vitamin A derivatives are only used as a comparison against retinyl linoleate. Specifically, these Vitamin A derivatives are retinol and hydroxypinacolone retinoate. In addition, all Vitamin A and Vitamin A derivatives presented in this invention are oil/lipid soluble. Therefore, a pre-mixture of these ingredients in the oil phase must be prepared in order to achieve a desirable finished formulation.

### Example 1

Preparation of oil in water emulsion with retinyl linoleate and tetrahexydecyl ascorbate/ascorbyl tetraisopalmitate combination.

A typical oil-in-water emulsion is used for incorporating the combination of Vitamin A and Vitamin C. Since the stability of the Vitamin combination in the presence of a finished formulation can be evaluated based on the characteristic odor, visual appearance, specific gravity, pH and viscosity, choosing an emulsion base with a white color background is favorable for monitoring these parameters. The oil-in-water emulsion is based on a technology utilized hydrogenated lecithin in order to improve the emulsion stability.

| INCI Name | % w/w |
|---|---|
| Phase A | |
| Water | 63.8 |
| Isopentyldiol | 5.00 |
| Glycerine | 2.00 |
| Xanthan Gum | 5.00 |
| Phenoxyethanol | 1.00 |
| Phase B | |
| Glyceryl Stearate | 2.50 |
| Cetyl Alcohol | 2.00 |
| Stearyl Alcohol | 2.00 |
| Macadamia Ternifolia Seed | 10.00 |
| Oil | |
| Isononyl Isononanoate | 3.00 |
| Hydrogenated Lecithin | 0.50 |
| Batyl Alcohol | 0.50 |
| Phase C | |
| Retinyl Linoleate | 0.2 |
| Tetrahexydecyl | 2.00 |
| Ascorbate/Ascorbyl | |
| Tetraisopalmitate | |
| Phase D | |
| Dimethicone | 0.50 |

### Procedure

1. The ingredients of phase A are combined and mixed at room temperature 25° C., until homogeneous. Then the combination is heated up to 75° C.-80° C.
2. The ingredients of phase B are combined and heated up to 75° C.-80° C. Keep mixing until homogeneous.
3. The ingredients of phase C are combined and added to phase B while the temperature is maintained at 75° C.-80° C.
4. The mixture of phase B and phase C is added to phase A slowly under homogenizer. Maintain the temperature at 75° C.-80° C. during addition and the speed of homogenizer is 5000 rpm.
5. The mixture is cooled down to 40° C. and phase D is slowly added into the mixture under sweeper blade. Then the whole mixture is cooled down to 25° C. under sweeper blade.

Stability assessment of the example formulation is critical. Monitoring the color change of the formulation as a function of time or temperature is a direct indication of the Vitamin A and Vitamin C stability. Both Vitamin A and Vitamin C are sensitive to the pH, temperature and oxygen.

However, the particular combination in this formulation example can be exposed to these environmental factors and maintain a high stability.

In terms of the stability assessment, monitoring the change of color is the most important method, and is completed frequently. Other physicochemical properties such as pH, specific gravity and viscosity are also important, but will only be measured at certain times. All properties will be determined using corresponding equipment.

### Example 2

Comparative example oil in water emulsion with retinyl linoleate and ascorbyl glucoside. The composition was prepared according to example 1, except that tetrahexydecyl ascorbate/ascorbyl tetraisopalmitate 2% in phase C is replaced with ascorbyl glucoside 2%. In the meantime, due to the temperature sensitivity of ascorbyl glucoside, a water solution with pH at 6 is made, then post added into the emulsion, after phase D.

### Example 3

Comparative example oil in water emulsion with retinyl linoleate and 3-O-ethyl ascorbic acid.

The composition was prepared according to example 1, except that tetrahexydecyl ascorbate/ascorbyl tetraisopalmitate 2% in phase C is replaced with 3-O-ethyl ascorbic acid 2%. In the meantime, due to the temperature sensitivity of 3-O-ethyl ascorbic acid, a water solution with pH at 6 is made then post added into the emulsion, after phase D.

### Example 4

Comparative example oil in water emulsion with retinyl linoleate and magnesium ascorbyl phosphate.

The composition was prepared according to example 1, except that tetrahexydecyl ascorbate/ascorbyl tetraisopalmitate 2% in phase C is replaced with magnesium ascorbyl phosphate 2%. In the meantime, due to the temperature sensitivity of magnesium ascorbyl phosphate, a water solution with pH at 6 is made then post added into the emulsion, after phase D.

### Example 5

Comparative example oil in water emulsion with retinol and tetrahexydecyl ascorbate/ascorbyl tetraisopalmitate.

The composition was prepared according to example 1, except that retinol linoleate 0.2% in phase C is replaced with retinol 0.2%. Moreover, retinol is post added as phase E after phase D due to its temperature sensitivity

### Example 6

Comparative example oil in water emulsion with retinol and ascorbyl glucoside.

The composition was prepared according to example 2, except that retinol linoleate 0.2% in phase C is replaced with retinol 0.2%. Moreover, retinol is post added as phase E after phase D due to its temperature sensitivity.

### Example 7

Comparative example oil in water emulsion with retinol and 3-O-ethyl ascorbic acid.

The composition was prepared according to example 3, except that retinol linoleate 0.2% in phase C is replaced with retinol 0.2%. Moreover, retinol is post added as phase E after phase D due to its temperature sensitivity.

### Example 8

Comparative example oil in water emulsion with retinol and magnesium ascorbyl phosphate.

The composition was prepared according to example 4, except that retinol linoleate 0.2% in phase C is replaced with retinol 0.2%. Moreover, retinol is post added as phase E after phase D due to its temperature sensitivity.

### Example 9

Comparative example oil in water emulsion with hydropinacolone retinoate and tetrahexydecyl ascorbate/ascorbyl tetraisopalmitate.

The composition was prepared according to example 1, except that retinol linoleate 0.2% in phase C is replaced with hydroxypinacolone retinoate 0.2%. Moreover, hydropinacolone retinoate is post added as phase E after phase D due to its temperature sensitivity.

### Example 10

Comparative example oil in water emulsion with hydropinacolone retinoate and ascorbyl glucoside.

The composition was prepared according to example 2, except that retinol linoleate 0.2% in phase C is replaced with hydroxypinacolone retinoate 0.2%. Moreover, hydropinacolone retinoate is post added as phase E after phase D due to its temperature sensitivity.

### Example 11

Comparative example oil in water emulsion with hydropinacolone retinoate and 3-O-ethyl ascorbic acid.

The composition was prepared according to example 3, except that retinol linoleate 0.2% in phase C is replaced with hydroxypinacolone retinoate 0.2%. Moreover, hydropinacolone retinoate is post added as phase E after phase D due to its temperature sensitivity.

### Example 12

Comparative example oil in water emulsion with hydropinacolone retinoate and magnesium ascorbyl phosphate.

The composition was prepared according to example 4, except that retinol linoleate 0.2% in phase C is replaced with hydroxypinacolone retinoate 0.2%. Moreover, hydropinacolone retinoate is post added as phase E after phase D due to its temperature sensitivity.

### Results and Discussion

Lecithin based oil-in-water emulsions were used as substrates to formulate with combination of Vitamin actives. In order to determine the interactions and stabilities of Vitamin actives, photos were taken periodically as a function of time. The emulsion base is white, and it is easy to observe any changes in color if the samples are closely monitored. As illustrated previously, the active Vitamins are Vitamin C and Vitamin A derivatives. Vitamin C derivatives used in the formulations are tetrahexyldecyl ascorbate/ascorbyl tetraisopalmitate, ascorbyl glucoside, 3-O-ethyl ascorbic acid and magnesium ascorbyl phosphate. Vitamin A derivatives are retinyl linoleate, retinol and hydroxypinacolone retinoate. Formulation with these ingredients strictly follow recommended formulation guidelines with consideration to pH, temperature and sequence of addition.

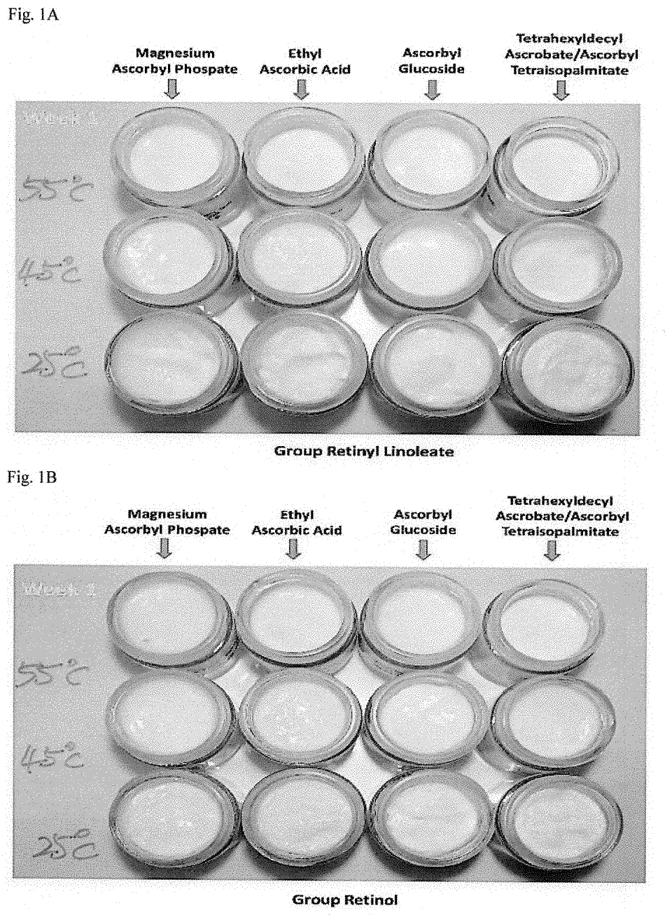

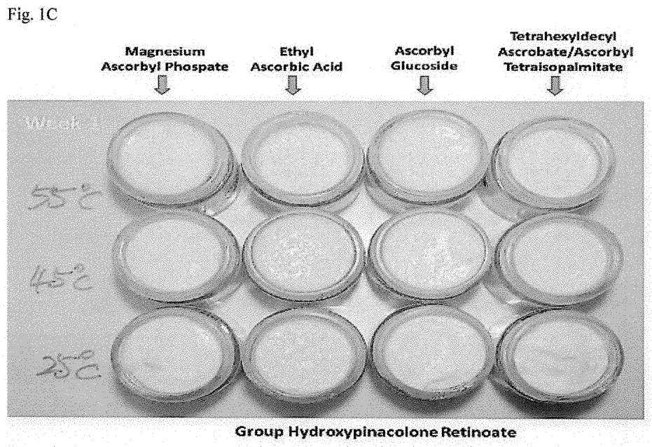

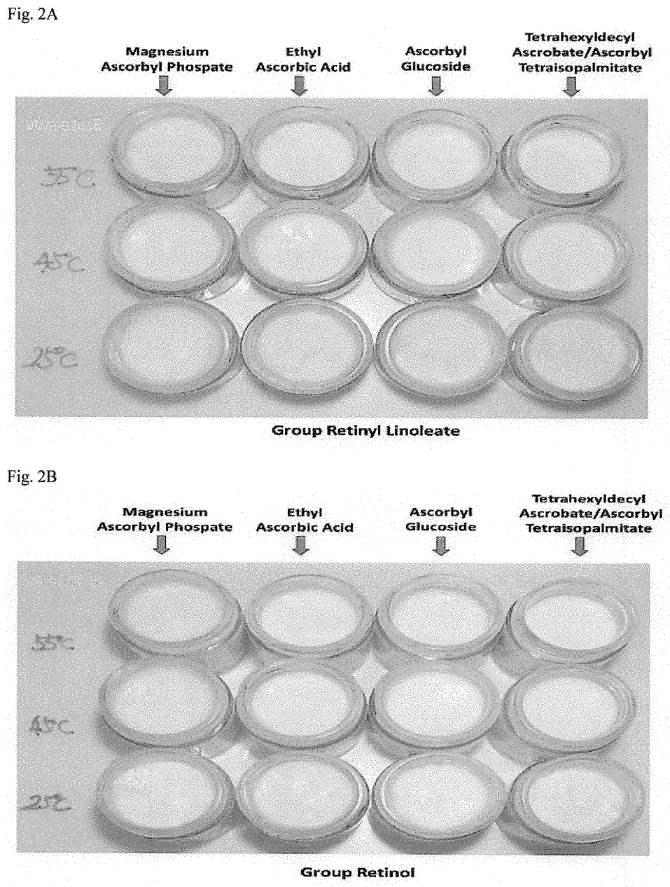

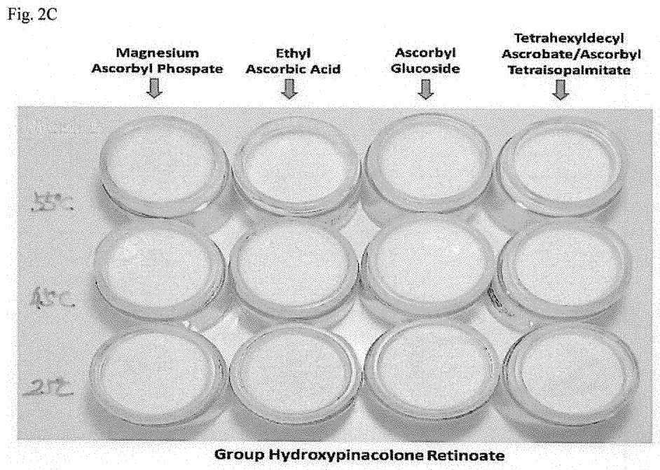

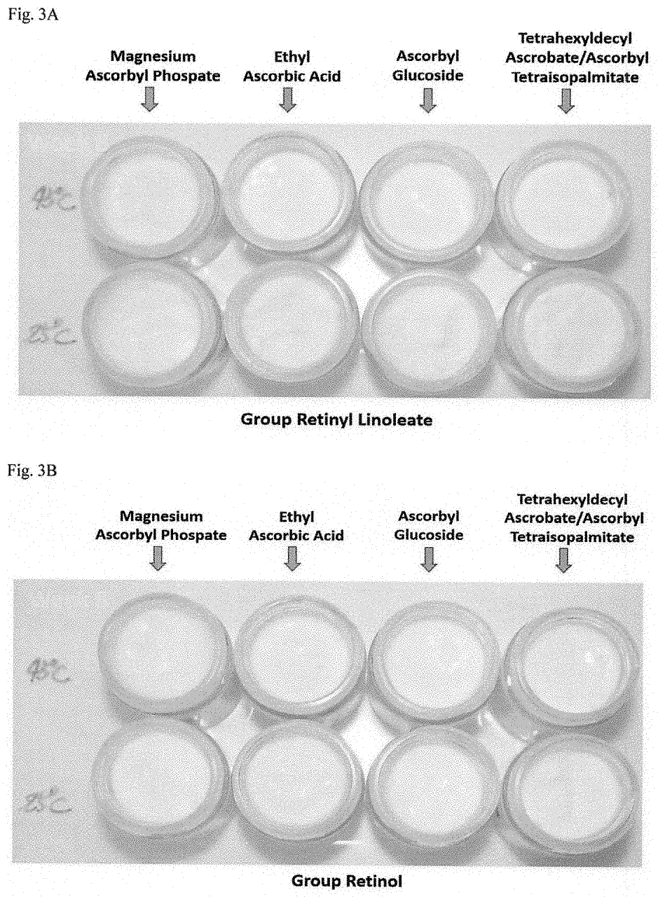

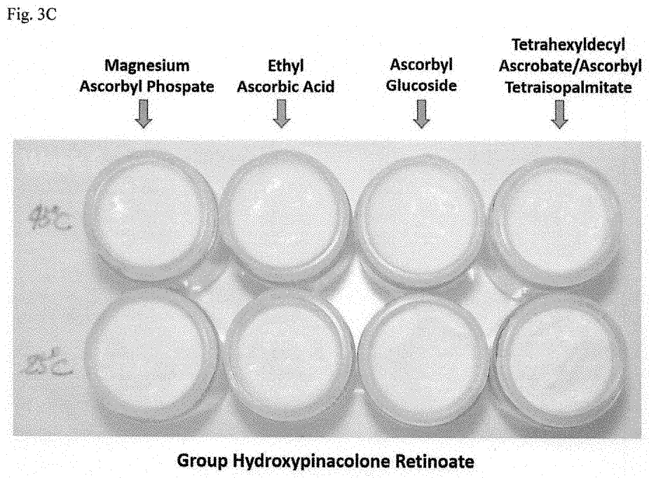

One of the most important criteria to determine the stability of the samples is the color change. Almost all skincare formulations have a high standard regarding color performance due to the importance of the customer user-experience. It is undesirable to observe visible color changes in cosmetic products. Example 1 through example 12 in total generate 12 samples with active combinations which differ from one another. Example 1 to example 4 are formulated with retinyl linoleate (labeled as “Group Retinyl Linoleate”) and four other Vitamin C derivatives. The representative samples at different temperatures of 25° C., 45° C. and 55° C. are displayed in three sample rows from bottom to top in FIG. 1A (week 1) and FIG. 2A (week 5). Likewise, samples from example 5 to example 8 are formulated with retinol (labeled as “Group Retinol”) and Vitamin C derivatives. The representative images are shown in FIG. 1B (week 1) and FIG. 2B (week 5). Moreover, representative samples from example 9 to example 12, formulated with hydroxypinacolone retinoate (labeled as “Group Hydroxypinacolone retinoate”) and four other Vitamin C derivatives are shown in FIG. 1C (week 1) and FIG. 2C (week 5). The INCI name of Vitamin C derivatives, tetrahexyldecyl ascorbate/ascorbyl tetraisopalmitate, ascorbyl glucoside, 3-O-ethyl ascorbic acid and magnesium ascorbyl phosphate (abbreviated as “Mg VC phosphate” in the figure), are labeled from left to right on top of each sample column in each figure.

Stability of Samples after One-Week Incubation at Varied Temperatures

All samples formulated with retinyl linoleate (FIG. 1A) are white after a one-week incubation at all temperatures (25° C., 45° C. and 55° C.) and the color did not change compared with the color before subjected to incubation. However, in FIG. 1B, samples formulated with 3-O-ethyl ascorbic acid and retinol became creamy yellow at 55° C. The combination of 3-O-ethyl ascorbic acid and retinol at 45° C. also turned yellow, but the color change is less intense than the 55° C. The rest of the samples are white and no significant change in color is observed. This phenomenon indicates the following: First, within the “Group Retinol” samples, the combination of 3-O-ethyl ascorbic acid and retinol is the least stable. Second, “Group Retinyl Linoleate” is more stable than “Group Retinol” due to the fact that no color change of the samples is observed through all incubation temperatures.

Despite the fact that hydroxypiancolone retinoate is an ester derivative of retinol, “Group Hydroxypinacolone Retinoate” showed a completely different color profile than samples formulated with retinyl linoleate and retinol (FIG. 1C). Samples formulated with hydroxypiancolone retinoate were all creamy yellow on the first day before incubation. This color profile is undesirable, especially when customers pursue a product with white or no color. As shown in FIG. 1C, the sample formulated with magnesium ascorbyl phosphate with hydroxypianocolone retinoate turned more yellow at 45° C. and 55° C. (55° C. the most) than the rest of the samples. This also indicates that the combination of magnesium ascorbyl phosphate and hydroxypianolone retinoate is not stable after one week of incubation at an elevated temperature. In summary, the major change in the ingredients from FIG. 1A to 1C is the Vitamin A derivative. It demonstrates that retinyl linoleate in combination with these four Vitamin C derivatives are the most stable in terms of color, and the combinations with retinol shows poor stability in terms of color. Moreover, combinations with hydroxypianolone retinoate are the least favorable in color presence, and the samples also show color instability.

Stability of Samples after Five and Eight-Week Incubation at Varied Temperatures

There was a more drastic change in color after five weeks of incubation, and the images are shown in FIG. 2A to 2C. Samples formulated with retinyl linoleate still remain white and only the samples incubated at 55° C. showed a traceable hint of yellow, except the one formulated with tetrahexydecyl ascorbate/ascorbyl tetraisopalmitate. The 45° C. and 25° C. samples shared the same color as in FIG. 1A. However, samples formulated with retinol exhibited significant color change after the incubation. Samples incubated at 25° C. showed a slight change in color from white to pale yellow. When the temperature increased to 45° C., samples became creamy yellow and the color change was more intense than the 25° C. samples. At the highest temperature, 55° C., samples turned lemony yellow. Particularly for the sample formulated with magnesium ascorbyl phosphate, it showed yellow-color inhomogeneity and patches on the top of the emulsion. This color change was attributed to the oxidation and instability of retinol, since such color change is not observed in the samples formulated with retinyl linoleate, using the same type of Vitamin C derivatives.

In regard to samples formulated with hydroxypinacolone retinoate, the color change was intense. In week one, the color of these samples was creamy yellow and after four weeks incubation, the color changed to lemony yellow. In the meantime, samples formulated with magnesium ascorbyl phosphate and hydroxypinacolone retinoate showed color inhomogeneity (FIG. 2C). Although it somewhat resembles the sample formulated with magnesium ascorbyl phosphate and retinol, the sample itself became phase-separated. It had a thick cream layer on the top, and a layer of transparent oil at the bottom. This combination broke the emulsion base. Based on the visual results from in FIG. 2A, the combination with retinyl linoleate and tetrahexydecyl ascorbate/ascorbyl tetraisopalmitate shows superior stability compared with other samples formulated with retinyl linoleate. When compared against FIG. 2B, samples formulated with retinyl linoleate were more stable than samples formulated with the other two Vitamin A's after five weeks. The difference was small in the first week's result, but it became much more significant four weeks later. In essence, the stability of samples formulated with retinyl linoleate is overall higher than the other two groups of samples.

Monitoring and accessing visual stability of these samples was also continued to week eight, which is two-month period. For samples incubated at 55° C., the stability monitoring that stopped at five weeks correlated to industrial standard (usually 4 weeks at 55° C.). Samples at 25° C. and 45° C. were monitored after five weeks. Results in week eight were similar with results in week five. Formulations with retinyl linoleate had the highest visual stability, especially the sample with tetrahexydecyl ascorbate/ascorbyl tetraisopalmitate and retinyl linoleate. Samples formulated with retinol and Vitamin C derivatives not only changed in color, but also showed phase separation. For example, the sample formulated with retinol and magnesium ascorbyl phosphate became more yellow, and the emulsion separated into two phases at 55° C. This result was similar to the sample formulated with hydroxypinacolone retinoate and magnesium ascorbyl phosphate at week five incubated at 55° C. Samples with other Vitamin C derivatives did not became phase separated. The above results indicate that the visual instability and emulsion stability are determined by both the type of Vitamin A, and the type of Vitamin C. Therefore, the combination of retinyl linoleate and tetrahexyldecyl ascorbate/ascorbyl tetraisopalmitate is one of the most stable among all combinations, and formulations with retinyl linoleate display the best visual profile and stability compared with formulations with the other two Vitamin A derivatives.

Although a specific embodiment of the invention has been disclosed herein, it is to be understood that various modifications can be made to the described embodiment without departing from the scope of the claimed invention, which modification would be apparent to one skilled in this art area.

What is claimed is:

1. A combination of Vitamin A and Vitamin C with enhanced stability that contains one Vitamin A derivative and one Vitamin C derivative selected from a group of Vitamin C derivatives.

2. A combination according to claim 1 wherein said Vitamin A derivative is retinyl linoleate.

3. A combination according to claim 1 wherein said Vitamin A derivative is in a carrier oil.

4. A combination according to claim 1 wherein said several other Vitamin C derivatives are selected from a group comprising tetrahexydecyl ascorbate/ascorbyl tetraisopalmitate, ascorbyl glucoside, 3-O-ethyl ascorbic acid and magnesium ascorbyl phosphate.

5. A combination according to claim 1 wherein the amount of retinyl linoleate ranges from 0.01% to 2% by weight.

6. A combination according to claim 4 wherein the amount of tetrahexydecyl ascorbate/ascorbyl tetraisopalmitate ranges from 0.01% to 2% by weight.

7. A combination according to claim 4 wherein the amount of ascorbyl glucoside ranges from 0.01% to 2% by weight.

8. A combination according to claim 4 wherein the amount of 3-O-ethyl ascorbic acid ranges from 0.01% to 2% by weight.

9. A combination according to claim 4 wherein the amount of magnesium ascorbyl phosphate ranges from 0.01% to 2% by weight.

10. A combination according to claim 1 wherein said final composition is in the form of a lotion, a cream, a milk, a gel, an oil, an aerosol, a spray, a foam, a solid stick, a powder, a shampoo, a hair conditioner, a lacquer, a make-up or a sunscreen.
